(19) United States
(12) Patent Application Publication
(10) Pub. No.: US 2012/0201791 A1
(43) Pub. Date: Aug. 9, 2012

(54) METHODS OF TREATING DISEASES OR CONDITIONS USING MESENCHYMAL STEM CELLS

(76) Inventor: Tai June Yoo, Collierville, TN (US)

(21) Appl. No.: 13/502,061

(22) PCT Filed: Oct. 15, 2010
(86) PCT No.: PCT/US10/52953

§ 371 Date: Apr. 13, 2012

Related U.S. Application Data
(60) Provisional application No. 61/252,129, filed on Oct. 15, 2009.

(51) Int. Cl. A61K 35/12; A61P 29/00; A61P 25/00; A61P 9/00; A61P 1/04; A61P 27/16; A61P 17/00; A61P 11/02; A61P 11/06; A61P 11/00; A61P 37/06; A61P 19/02; A61P 17/06

(52) U.S. Cl. 424/93.7

(57) ABSTRACT

The present invention provides a method of treating or preventing a disease or condition in a patient comprising intravenously administering a therapeutically effective amount of mesenchymal stem cells to the patient, wherein the disease or condition is osteoarthritis, rheumatoid arthritis, multiple sclerosis, stroke, ulcerative colitis, psoriasis, Hashimoto's thyroiditis, atopic dermatitis, allergic rhinitis, chronic obstructive pulmonary disease with bronchial asthma or hearing loss.

### CROSS REFERENCE TO RELATED APPLICATIONS

This application claims the benefit of U.S. Appl. No. 61/252,129, filed Oct. 15, 2009. The content of the aforesaid application is relied upon and incorporated by reference in its entirety.

### FIELD OF THE INVENTION

The field of the invention relates to stem cells and regenerative medicine. More specifically, the field of the invention relates to the use of mesenchymal stem cells, in particular, mesenchymal stem cells of adipose origin, to treat various diseases or conditions in a patient. The field of the invention also relates to the use of mesenchymal stem cells to treat hearing loss in a patient.

### BACKGROUND OF THE INVENTION

Stem cells refer to cells having not only self-replication ability but also the ability to differentiate into at least two cells, and can be divided into totipotent stem cells, pluripotent stem cells, and multipotent stem cells.

Totipotent stem cells are cells having totipotent properties capable of developing into one perfect individual, and these properties are possessed by cells up to the 8-cell stage after the fertilization of an oocyte and a sperm. When these cells are isolated and transplanted into the uterus, they can develop into an individual.

Pluripotent stem cells, which are cells capable of developing into various cells and tissues derived from the ectodermal, mesodermal and endodermal layers, are derived from an inner cell mass located inside of blastocysts generated 4-5 days after fertilization. These cells are called “embryonic stem cells” and can differentiate into various other tissue cells but not form new living organisms.

Multipotent stem cells, which are stem cells capable of differentiating into only cells specific to tissues and organs containing these cells, are involved not only in the growth and development of various tissues and organs in the fetal, neonatal and adult periods but also in the maintenance of homeostasis of adult tissue and the function of inducing regeneration upon tissue damage. Tissue-specific multipotent cells are collectively called “adult stem cells.”

Adult stem cells are obtained by collecting cells from various human organs and developing the cells into stem cells and are characterized in that they differentiate into only specific tissues. However, recently, experiments for differentiating adult stem cells into various tissues, including liver cells, have been dramatically successful.

The multipotent stem cells were first isolated from adult bone marrow (Jiang et al., Nature, 418:41, 2002), and then also found in other various adult tissues (Verfaillie, Trends Cell Biol., 12:502, 2002). In other words, although the bone marrow is the most widely known source of stem cells, the multipotent stem cells were also found in the skin, blood vessels, muscles and brains (Tomas et al., Nat. Cell Biol., 3:778, 2001; Sampaolesi et al., Science, 301:487, 2003; Jiang et al., Exp. Hematol., 30:896, 2002). However, stem cells in adult tissues, such as the bone marrow, are very rarely present and such cells are difficult to culture without inducing differentiation, and thus difficult to culture in the absence of specifically screened media. It is very difficult to maintain the isolated stem cells in vitro.

Recently, adipose tissue was found to be a new source of multipotent stem cells (Cousin et al., BBRC., 301:1016, 2003; Miranville et al., Circulation, 110:349, 2004; Gronthos et al., J. Cell Physiol., 189:54, 2001; Seo et al., BBRC., 328:258, 2005). It was reported that a group of undifferentiated cells is included in human adipose tissue obtained by liposuction and has the ability to differentiate into fat cells, osteogenic cells, myoblasts and chondroblasts (Zuk et al., Tissue Eng., 7:211, 2001; Rodriguez et al., BBRC., 315:255, 2004). Also, recent studies using animal model experiments indicate that adipose tissue-derived cells have the abilities to regenerate muscles and to stimulate the differentiation of nerve blood vessels.

A discussion of mesenchymal stem cells inhibiting pathological immunity is described in Aggarwal et al., Blood, 105:1815-22 (2005). There is a need for new and effective treatments for diseases using stem cells for which traditional therapies have failed or have significant shortcomings, including toxicities, resistance or other unwanted side effects. Surprisingly, the present inventor has discovered that autologous mesenchymal stem cells, in particular mesenchymal stem cells derived from adipose tissue are effective in the treatment of various diseases and conditions.

### SUMMARY OF THE INVENTION

In one aspect, the present invention provides a method of treating or preventing a disease in a patient comprising administering intravenously a therapeutically effective amount of mesenchymal stem cells to the patient, wherein the disease is selected from the group consisting of osteoarthritis, rheumatoid arthritis, hearing loss, multiple sclerosis, stroke, ulcerative colitis, Hashimoto's thyroiditis, atopic dermatitis, psoriasis, allergic rhinitis, and chronic obstructive pulmonary disease with bronchial asthma.

In another aspect, the present invention provides a method of treating or preventing a disease in a patient comprising administering intravenously a therapeutically effective amount of adipose tissue derived mesenchymal stem cells to the patient, wherein the disease is selected from the group consisting of osteoarthritis, rheumatoid arthritis, hearing loss, multiple sclerosis, stroke, ulcerative colitis, Hashimoto's thyroiditis, atopic dermatitis, psoriasis, allergic rhinitis, and chronic obstructive pulmonary disease with bronchial asthma.

In another aspect, the present invention provides a method of treating or preventing autoimmune hearing loss in a patient comprising administering intravenously a therapeutically effective amount of mesenchymal stem cells to the patient.

In another aspect, the present invention provides a method of treating or preventing hearing loss in a patient comprising administering intravenously a therapeutically effective amount of adipose tissue derived mesenchymal stem cells to the patient.

### BRIEF DESCRIPTION OF THE FIGURES

Embodiments of the invention are herein described, by way of non-limiting example, with reference to the following accompanying Figures:

FIG. 1. Adult stem cells.

FIG. 2. Treatment of Atopic dermatitis using autologous mesenchymal stem cells from adipose tissue.

FIG. 3A-B. Restoration of hearing in a mouse model of autoimmune inner ear disease using adipose mesenchymal stem cells.

FIG. 4. Splenocytes from the mice that were administered human adipose derived mesenchymal stem cells (AD-MSCs) produced significantly lower levels of IL-17 and IFN-γ than did cells from mice administered PBS. Human AD-MSCs dramatically stimulated the production of IL-10 by β-tubulin-activated T cells, whereas the Th2-type cytokine IL-4 was not significantly affected.

FIG. 5. Administration of human AD-MSCs had significantly higher numbers of CD4⁺CD25⁺FoxP3⁺ Treg cells in splenocytes (A) than did PBS control mice (B), indicating human AD-MSCs could be inducing Treg cells secreting IL-10, which suppresses the self-reactive T cells.

FIG. 6. Treatment with human adipose-derived mesenchymal stem cells (AD-MSCs) prevents and suppresses collagen-induced arthritis.

### DETAILED DESCRIPTION OF THE INVENTION

It is to be understood that this invention is not limited to the particular methods, compositions and materials disclosed herein as such methods, compositions and materials may vary. It is also understood that the terminology employed herein is used for the purpose of describing particular embodiments only and is not intended to be limiting since the scope of the present invention will be limited only by the appended claims and equivalents thereof.

Reference will now be made in detail to the following embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, the preferred methods, devices, and materials now described.

Providing a therapy or “treating” refers to indicia of success in the treatment or amelioration of an injury, disease or condition, including any objective or subjective parameter such as abatement, remission, diminishing of symptoms of making the injury, disease or condition more tolerable to the patient, slowing the rate of degeneration or decline, making the final point of degeneration less debilitating, or improving a patient's physical or mental well-being. Those in need of treatment include those already with the disease or condition as well as those prone to have the disease or condition or those in whom the disease or condition is to be prevented. Preferred subject for treatment include animals, most preferably mammalian species, such as humans and domestic animals such as dogs, cats, and the like, subject to the disease and other conditions. A “patient” refers to a subject, preferably mammalian (including human). Where the specification indicates that a number of cells are to be administered, a person of ordinary skill in the art will understand that these are approximate values.

It must also be noted that, as used in this specification and the appended claims, the singular forms “a,” “an,” and “the” include plural referents unless the context clearly dictates otherwise.

As used herein, the term “stem cells” refers to cells that can reproduce indefinitely to form the specialized cells of tissues and organs. Stem cells are developmental pluripotent or multipotent cells. Stem cells can divide to produce two daughter stem cells, or one daughter stem cell and one progenitor (“transit”) cell, which then proliferates into fully differentiated and mature cells in tissue.

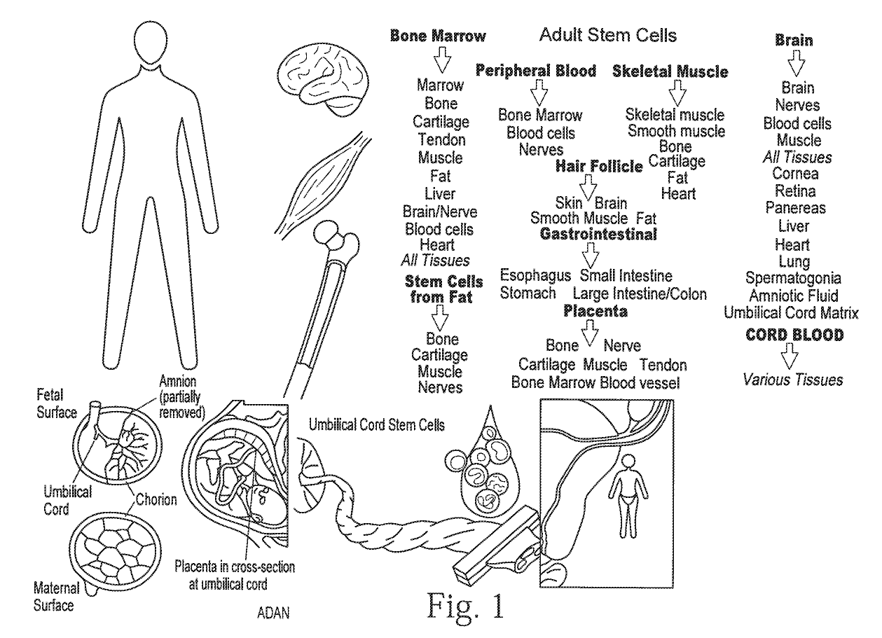

Mesenchymal stem cells are multipotent stem cells that can differentiate into a variety of cell types. Cell types that mesenchymal stem cells have been shown to differentiate into in vitro or in vivo include osteoblasts, chondrocytes, myocytes, adipocytes, endotheliums, and beta-pancreatic islets cells. Mesenchymal stem cells can be obtained from bone marrow, placental matrix, adipose tissue and cord blood, for example (see FIG. 1). These cells, once isolated are expandable in large numbers, regenerative and immune modulatory (hypo immunogenic).

Mesenchymal stem cells are shown to suppress immune reactions both in vitro and in vivo in a non-MHC restricted manner.

The term “adipose tissue” used herein refers to tissue including plural cytotypes such as an adipocyte, a microvascular cell and the like. Also, the adipose tissue includes connective tissue storing adipose.

In one embodiment, the present invention provides a method of treating or preventing a disease in a patient comprising intravenously administering a therapeutically effective amount of mesenchymal stem cells to the patient, wherein the disease is selected from the group consisting of osteoarthritis, rheumatoid arthritis, autoimmune hearing loss, multiple sclerosis, stroke, ulcerative colitis, Hashimoto's thyroiditis, atopic dermatitis, psoriasis, allergic rhinitis, and chronic obstructive pulmonary disease with bronchial asthma.

In some embodiments, the mesenchymal stem cells are autologous, meaning from the individual to be treated. In some embodiments, the autologous cells can be used in their natural state, or they can be modified genetically so that they express or do not express a desired gene before they are introduced into the patient. In some embodiments, they are propagated and expanded outside of the body before administration.

In some embodiments, autologous mesenchymal stem cells are isolated from the patient's adipose tissue. The adipose tissue may be obtained by a predetermined method well known to those of ordinary skill in the art. For example, the adipose tissue may be obtained by suction-assisted liposuction, ultrasonic-assisted liposuction, adipose tissue removal or combinations thereof. In suction-assisted liposuction, the adipose tissue is collected by inserting a cannula in or around an adipose tissue storage existing in a patient and sucking out lipids into suction equipment. Adipose tissue removal includes the steps of incidentally collecting tissue containing adipose tissue (e.g., skin), that is, target tissue for an operation (e.g., skin in lipectomy or cosmetic surgery) together with the adipose tissue.

In some embodiments, the cells are harvested through liposuction. The mesenchymal stem cells adhere to a culture flask and are capable of propagation and expansion in vitro. Other methods of isolation from adipose tissue are encompassed by the present invention. The adipose derived mesenchymal stem cells as used in the present invention are described in WO 2008/147057, herein incorporated by reference.

The isolation and culture of the mesenchymal stem cells from adipose tissue can be performed by any suitable method. In one embodiment, the mesenchymal stem cells can be isolated by the steps of treating collagenase to the adipose tissue at a sufficient concentration, such as for example, 1 mg/ml, culturing the adipose tissue in appropriate conditions (temperature and time), isolating floating fat cells by centrifugation or another method well known to those of ordinary skill in the art, and tissue-culturing precipitating stromal fractions.

The isolated mesenchymal stem cells can be cultured in a cell culture medium well known to those of ordinary skill in the art, which can include DMEM medium, McCoys 5 A medium (Gibco), Eagle's basal medium, CMRL media, Glasgow minimal essential medium, Ham's F-12 medium, Iscove's modified Dulbecco's medium, Liebovitz's L-15 medium, and RPMI 1640 medium, but the present invention is not limited thereto. Also, in some embodiments, at least one auxiliary element can be added when required, which can include: serum of calf, horse and human; antibiotics such as streptomycin sulfate and penicillin G for preventing contamination of microorganisms; and antifungal agents such as amphotericin B, gentamicin and nystatin.

In some embodiments, the isolated mesenchymal stem cells can be stored by a method well known to those of ordinary skill in the art before use. Generally, the mesenchymal stem cells can be cold-stored after cyroprotection treatment. The cyroprotection treatment can be performed using a cyroprotective agent such as dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose or choline chloride.

In an embodiment of the present invention, the adipose tissue-derived mesenchymal cells can be obtained by infusing a tumescent solution and a fat-containing material using liposuction tubing or a disposable syringe having a catheter connected thereto, subjecting the resulting materials to a mycoplasma test and a sterility test, selecting a sample from among the tested materials, centrifuging the selected sample into an adipose layer and an aqueous layer, pre-treating the aqueous layer sample with a collagenase solution, and then culturing the resulting cells in a DMEM medium containing 10% FBS and ascorbic acid.

In some embodiments, methods for obtaining multipotent stem cells expressing desired surface antigens from the human adipose tissue-derived stern cell broth obtained above include a FACS method using a flow cytometer with sorting function (Int. Immunol, 10(3):275, 1998), a method using magnetic beads, and a panning method using an antibody specifically recognizing multipotent stem cells (J. Immunol, 141(8):2797, 1998). Also, methods for obtaining multipotent stem cells from a large amount of culture broth include a method where antibodies, which are expressed on the surface of cells to specifically recognize molecules (hereinafter, referred to as “surface antigens”), are used alone or in combination as columns.

Flow cytometry sorting methods may include a water drop charge method and a cell capture method. In any of these methods, an antibody specifically recognizing an antigen on the cell surface is fluorescently labeled, the intensity of fluorescence emitted from an antibody bonded with the molecule expressed on the surface of the cell is converted to an electric signal whereby the expressed amount of the antigen can be quantified. It is also possible to separate cells expressing a plurality of surface antigens by combination of fluorescence types used therefor. Examples of fluorescences which can be used in this case include FITC (fluorescein isothiocyanate), PE (phycoerythrin), APC (allo-phycocyanin), TR (Texas Red), Cy 3, CyChrome, Red 613, Red 670, TRI-Color, Quantum Red, etc.

FACS methods using a flow cytometer include: a method where the above stem cell broth is collected, from which cells are isolated by, for example, centrifugation, and stained directly with antibodies; and a method where the cells are cultured and grown in a suitable medium and then stained with antibodies. The staining of cells is performed by mixing a primary antibody recognizing a surface antigen with a target cell sample and incubating the mixture on ice for 30 minutes to 1 hour. When the primary antibody is fluorescently labeled, the cells are isolated with a flow cytometer after washing. When the primary antibody is not fluorescently labeled, cells reacted with the primary antibody and a fluorescent labeled secondary antibody having binding activity to the primary antibody are mixed after washing, and incubated on ice water for 30 minutes to 1 hour. After washing, the cells stained with the primary and secondary antibodies are isolated with a flow cytometer.

In some embodiments, the isolated mesenchymal stem cells for use in the present invention can be analyzed for their immunological properties using flow cytometry. In some embodiments, the adipose tissue-derived stem cells for use in the present invention showed positive responses to CD73, CD90, CD29, CD44, and CD105. In some embodiments, the stem cells are from adults.

In some embodiments, the adipose tissue-derived stem cells for use in the present invention show positive responses of 91% to CD73, 97% to CD90, 96% to CD29, 83% to CD44, and 80% to CD105. In some embodiments, the mesenchymal stem cells are negative for CD31, CD34 and CD45. Also, in some embodiments, the mesenchymal stem cells showed negative immunological responses to all of CD33, CD34, CD45, CD4, CD31, CD62p, CD14 and HLA-DR.

In some embodiments, the cell therapeutic composition of mesenchymal stem cells can be administered at one or more sites, including local or systemic administration, or both. In some embodiments, the mesenchymal stem cell therapeutic compositions for use in the present invention can be introduced intravenously alone, or intravenously in combination with local administration (injection) at the site of condition to be treated. In some embodiments, the cells can be formulated with a pharmaceutically acceptable carrier.

In some embodiments, the dosage of the composition encompassing a therapeutically effective amount of mesenchymal stem cells ranges from 1.0×10³-1.0×10⁸ cells/kg (weight). In some embodiments, the dosage ranges from 1.0×10⁴-1.0×10⁷ cells/kg (weight). In some embodiments, about 1.0×10³ cells/kg, about 1.0×10⁴ cells/kg, about 1.0×10⁵ cells/kg, about 1.0×10⁶ cells/kg, about 1.0×10⁷ cells/kg or about 1.0×10⁸ cells/kg are administered.

The dosage of the composition may vary depending on patient's weight, age, sex and symptoms, the dosage form of the composition to be administered, a method of administering the composition, and so on. The frequency of administration may range from one to several times. There may be one or more administration sites. The dosage per kg for nonhuman animals may be the same as that for human, or can be converted from the above-described dosage, for example, based on the volume ratio (for example, average value) between the diseased tissue of the human and animal subjects. Animals to be treated according to the present invention include human and other desired mammals, specific examples of which include humans, monkeys, mice, rats, rabbits, sheep, horses, cats, cows and dogs.

In accordance with the present invention, the diseases or conditions can be treated or prevented by intravenous administration of the mesenchymal stem cells described herein. In some embodiments, about 20 million, about 40 million, about 60 million, about 80 million, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, about 500 million, about 520 million, about 540 million, about 560 million, about 580 million, about 600 million, about 620 million, about 640 million, about 660 million, about 680 million, about 700 million, about 720 million, about 740 million, about 760 million, about 780 million, about 800 million, about 820 million, about 840 million, about 860 million, about 880 million, about 900 million, about 920 million, about 940 million, about 960 million, or about 980 million cells are injected intravenously. In some embodiments, about 1 billion, about 2 billion, about 3 billion, about 4 billion or about 5 billion cells or more are injected intravenously. In some embodiments, the number of cells ranges from between about 20 million to about 4 billion cells, between about 40 million to about 1 billion cells, between about 60 million to about 750 million cells, between about 80 million to about 400 million cells, between about 100 million to about 350 million cells, and between about 175 million to about 250 million cells.

In some embodiments, a single intravenous administration is sufficient, while in other embodiments, multiple intravenous administrations are performed, such as 2, 3, 4, 5, 6, 7, 8, 9 10, 11, 12, 13, 14, 15, 16, 17, 18, 19, or 20 intravenous administrations of the mesenchymal stem cells. The treatment interval(s) can be spaced such that an administration follows a prior administration by one day, 2 days, 3 days, 4 days, 5 days, 6 days, one week, 1½-2 weeks, 3 weeks, one month, or 2-3 months, 6 months, one year, or two years or longer. In some embodiments, the treatment interval is spaced in accordance with the progression of the patient's improvement or response to treatment. For example, in some embodiments, a first treatment is administered followed by a second treatment one week later, followed by a third treatment one month later, followed by a fourth treatment 6 months later.

In some embodiments, osteoarthritis or rheumatoid arthritis is treated by intravenous administration of the mesenchymal stem cells alone, or in some embodiments, in combination with injection into inter phalangeal joint spaces. In some embodiments, only local injections into the inter phalangeal joint spaces are carried out. In some embodiments, three intravenous injections are made, each containing about 200 million cells in combination with injection of about 40 million cells divided amongst the inter phalangeal joint spaces. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments, intravenous treatments are made every week, and the injections into the inter phalangeal joint spaces occur on the last day of intravenous treatment or shortly thereafter. In some embodiments the inter phalangeal treatment occurs 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days after the last day of intravenous treatment. In some embodiments, the inter phalangeal treatment interval occurs over several days, and includes multiple injections. In some embodiments, about 10 million, about 20 million, about 30 million, about 40 million, about 50 million, about 60 million, about 70 million, about 80 million, about 90 million or about 100 million additional cells are injected, divided among the joint spaces.

In some embodiments, Hashimoto's thyroiditis is treated by intravenous administration of the mesenchymal stem cells described herein. In some embodiments, three intravenous injections are made, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments, intravenous treatments are made every week, every 2 weeks, every 3 weeks, or every 4 weeks.

In some embodiments, ulcerative colitis is treated by intravenous administration of the mesenchymal stem cells described herein. In some embodiments, three intravenous injections are made, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments, intravenous treatments are made every week, every 2 weeks, every 3 weeks, or every 4 weeks. In some embodiments, ulcerative colitis may be associated with other diseases such as osteoarthritis, and the intravenous treatments in accordance with the invention are sufficient to treat both conditions.

In some embodiments, atopic dermatitis is treated by intravenous administration of the mesenchymal stem cells described herein. In some embodiments the atopic dermatitis is associated with allergic rhinitis or other allergic conditions, such as food allergies or dust mite allergies, for example, which is also treated by intravenous administration. In some embodiments, three intravenous injections are made, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments about 600 million, about 700 million, about 800 million, about 900 million, about 1 billion, about 2 billion, about 3 billion, about 4 billion, about 5 billion, about 6 billion or about 10 billion cells are injected intravenously. In some embodiments, intravenous treatments are made every week, every 2 weeks, every 3 weeks, or every 4 weeks. In accordance with the invention, both the atopic dermatitis and allergic rhinitis symptoms or other allergic symptoms that might be present are also treated.

In some embodiments, allergic rhinitis is treated by intravenous administration of the mesenchymal stem cells described herein. In some embodiments, three intravenous injections are made, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments about 600 million, about 700 million, about 800 million, about 900 million, about 1 billion, about 2 billion, about 3 billion, about 4 billion, about 5 billion, about 6 billion or about 10 billion cells are injected intravenously. In some embodiments, intravenous treatments are made every week, every 2 weeks, every 3 weeks, or every 4 weeks.

In some embodiments, chronic obstructive pulmonary disease with bronchial asthma is treated by intravenous administration of the mesenchymal stem cells described herein. In some embodiments, chronic obstructive pulmonary disease symptoms improve and the bronchial asthma symptoms disappear completely. In some embodiments, a single intravenous injection is performed containing about 300-400 million cells. In some embodiments, three intravenous injections are made, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously, in some embodiments, intravenous treatments are made every week, every 2 weeks, every 3 weeks, or every 4 weeks.

In some embodiments, hearing loss is treated by intravenous administration anti/or injection into the ear of the mesenchymal stem cells described herein. In some embodiments, the hearing loss is autoimmune hearing loss. In some embodiments, the hearing loss is noise-induced hearing loss. In some embodiments, the hearing loss is drug-induced hearing loss. In some embodiments, the hearing loss is progressive or is age-related. In some embodiments, the hearing loss is due to injury. In some embodiments, three intravenous injections are made in one week intervals, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments, mesenchymal stem cells are injected directly into the ear, such as the inner and/or middle ear. Such administrations can be made alone, or in combination with intravenous administrations. In some embodiments, about 100,000, about 250,000, about 500,000, about 1 million, about 2 million, about 3 million, about 4 million, about 5 million, about 7 million, about 10 million, about 20 million, about 30 million, about 40 million, about 50 million, about 60 million, about 70 million, about 80 million, about 90 million or about 100 million mesenchymal stem cells are injected into the middle or inner ear. In some embodiments, the injections into the inner and/or middle ear accompany or follow the intravenous injections. In some embodiments, intravenous treatments are made every week, and the injections into the inner and/or middle ear occur on the last day of intravenous treatment, in some embodiments the injections into the inner and/or middle ear occur 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days after the last day of intravenous treatment. In some embodiments, the inner and/or middle ear treatment interval occurs over several days, and includes multiple injections.

In some embodiments, multiple sclerosis is treated by intravenous administration of the mesenchymal stem cells alone, or in some embodiments, in combination with intrathecal injection. In some embodiments, only intrathecal injections are carried out. In some embodiments, between three to six intravenous injections are made, each containing about 180 million cells in combination with between three to six intrathecal injections of about 20-40 million cells each. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments, intravenous treatments are made every week, and the intrathecal injections occur on the last day of intravenous treatment or shortly thereafter. In some embodiments the intrathecal treatment occurs 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days after the last day of intravenous treatment. In some embodiments, the intrathecal treatment interval occurs over several days, and includes multiple injections. In some embodiments, about 10 million, about 20 million, about 30 million, about 40 million, about 50 million, about 60 million, about 70 million, about 80 million, about 90 million or about 100 million additional cells are injected intrathecal. In some embodiments, the first number of intravenous injections are made in weekly intervals and the intrathecal injections are made concurrently with the intravenous injections, and several months later, such as for example 1, 2, 3, 4, 5, 6, 7, 8, 9, 10, 11, or 12 months later, a second number of concurrent intravenous and intrathecal injections can be made, at weekly intervals. For example, in some embodiments, three concurrent intravenous and intrathecal injections are given in weekly intervals, followed by three additional concurrent intravenous and intrathecal injections in weekly intervals seven months later.

In some embodiments, stroke is treated by intravenous administration of the mesenchymal stem cells described herein. In some embodiments, three intravenous injections are made, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments about 600 million, about 700 million, about 800 million, about 900 million, about 1 billion, about 2 billion, about 3 billion, about 4 billion, about 5 billion, about 6 billion or about 10 billion cells are injected intravenously. In some embodiments, intravenous treatments are made every week, every 2 weeks, every 3 weeks, or every 4 weeks. In some embodiments, intrathecal and/or intraventricular injections are also carried out in addition to intravenous injections. In some embodiments, three to six intrathecal and/or intraventricular injections of about 20-40 million cells each are made. The intrathecal and/or intraventricular injections can be made concurrently with the intravenous injections, or can be made at a period before or after the intravenous injection.

In some embodiments, psoriasis is treated by intravenous administration of the mesenchymal stem cells described herein. In some embodiments, two to four intravenous injections are made, depending on the patient's response, each containing about 200 million cells. In some embodiments, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, or about 500 million cells are injected intravenously. In some embodiments about 600 million, about 700 million, about 800 million, about 900 million, about 1 billion, about 2 billion, about 3 billion, about 4 billion, about 5 billion, about 6 billion or about 10 billion cells are injected intravenously. In some embodiments, intravenous treatments are made every week, every 2 weeks, every 3 weeks, or every 4 weeks.

The therapeutic methods of the present invention can be conducted alone or in combination with other standard or advanced methods or pharmaceutical treatments.

The therapeutic composition of mesenchymal stem cells for use in the methods of the present invention can comprise pharmaceutically acceptable carriers and/or additives. Examples thereof include sterilized water, physiological saline, a standard butler (e.g., phosphoric acid, citric acid, or other organic acids), a stabilizer, salt, an antioxidant (e.g., ascorbic acid), a surfactant, a suspending agent, an isotonic agent, or a preservative. As used herein, the term “base” refers to a base solution in which the mesenchymal stem cells in the cell therapeutic composition are suspended. In some embodiments, physiological saline, phosphate buffered saline or Hartman-D (Choongwae Pharma Corp.) is used as the base solution.

In some embodiments, the cell therapeutic composition is prepared in a dosage form suitable for injection. In some embodiments, the mesenchymal stem cells are dissolved (suspended) in a pharmaceutically acceptable aqueous solution, or frozen in a solution state. The kit of the present invention may further comprise a desired pharmaceutically acceptable carrier that can be used to suspend or dilute the mesenchymal stem cells. Examples of such a carrier include distilled water, physiological saline, PBS and the like.

The composition for use in the present invention can contain a pharmaceutically acceptable carrier or excipient, or any necessary stabilizer or adsorption-preventing agent to provide a pharmaceutical preparation that is suitable for administration to humans or animals. The composition of the present invention can be formulated in the form of an injectable solution (e.g., injection solutions for subcutaneous, intradermal, intramuscular, intravenous and intraperitoneal injection). In some embodiments, upon the injection of the composition of mesenchymal stem cells, an analgesic agent, which can relieve pains, may be used.

The cell therapeutic composition of mesenchymal stem cells for use in the present invention can be filled into a syringe, a device, a cryovial in which cells can be frozen, or a pyrogen-free glass vial comprising rubber stoppers and aluminum caps, which contains liquid drugs.

The cell therapeutic composition of mesenchymal stem cells for use in the present invention can, if necessary, contain at least one selected from among suspending agent, solubilizing agents, stabilizers, isotonic agents, preservatives, adsorption-preventing agents, surfactants, diluents, vehicles, pH-adjusting agents, analgesic agents, buffering agents, sulfur-containing reducing agents and antioxidants, depending on the administration mode or formulation thereof.

Examples of the suspending agents may include methylcellulose, Polysorbate 80, hydroxyethylcellulose, gum acacia, gum tragacanth powder, sodium carboxymethylcellulose, polyoxyethylene sorbitan monolaurate, etc. The solubilizing agents include polyoxyethylene hydrogenated castor oil, polysorbate 80, nicotinamide, polyoxyethylene sorbitan monolaurate, Macrogol and castor oil fatty acid ethyl esters. The stabilizers include dextran 40, methylcellulose, gelatin, sodium sulfite, sodium metasulfite, etc. Examples of the isotonic agents are D-mannitol and sorbitol.

Examples of the preservatives include methyl parahydroxybenzoate, ethyl parahydroxybenzoate, sorbic acid, phenol, cresol, and chlorocresol. Examples of the adsorption preventing agents include human serum albumin, lecithin, dextran, ethylene oxide-propylene oxide copolymer, hydroxypropylcellulose, methylcellulose, polyoxyethylene hydrogenated castor oil, and polyethylene glycol.

The sulfur-containing reducing agents include N-acetylcysteine, N-acetylhomocysteine, thioctic acid, thiodiglycol, thioethanolamine, thioglycerol, thiosorbitol, thioglycolic acid and salts thereof, sodium thiosulfate, glutathione, and sulfhydryl-containing compounds such as thioalkanoic acid having 1 to 7 carbon atoms.

The antioxidants include, for example, erythorbic acid, dibutylhydroxytoluene, butylhydroxyanisole, [alpha]-tocopherol, tocopherol acetate, L-ascorbic acid and salts thereof, L-ascorbyl palmitate, L-ascorbyl stearate, sodium bisulfite, sodium sulfite, triamyl gallate, propyl gallate or chelating agents such as disodium ethylenediamine tetraacetate (EDTA), sodium pyrophosphate and sodium metaphosphate. The cryopreservatives include, for example, DMSO, glycerol, etc.

Furthermore, in some embodiments, the cell therapeutic composition of mesenchymal stem cells for use in the methods of the present invention can comprise conventional additives, such as inorganic salts, including sodium chloride, potassium chloride, calcium chloride, sodium phosphate, potassium phosphate and sodium hydrogen carbonate, and organic salts, including sodium citrate, potassium citrate and sodium acetate.

In some embodiments, sucrose or albumin is added to the mesenchymal stem cells to improve stability, prior to cold storage of the cells. In some embodiments, the cells are combined with physiological saline, sucrose, albumin and cryopreservative DMSO prior to freezing and cold storing the cells.

All publications, patents, and pre-grant patent application publications cited in this specification are herein incorporated by reference. In the case of inconsistencies, the present disclosure will prevail. The present invention will be further illustrated by the following non-limiting examples. These Examples are provided to aid in the understanding of the invention and are not to be construed as a limitation thereof.

### EXAMPLES

### Example 1

Isolation of human mesenchymal stem cells from adipose tissue. Human adipose tissues were obtained by simple liposuction from the abdominal subcutaneous fat of donor. The subcutaneous adipose tissues were digested with 4 ml RTase (RNLBIO, SEOUL, KOREA) per 1 g fat under gentle agitation for 60 min at 37 degrees Celcius (or 1 mg/ml collagenase type I (Gibco, Carlsbad, Calif.) under gentle agitation for 60 min at 37° C.). Next, the digested tissues were filtered through a 100 μm nylon sieve to remove cellular debris and centrifuged at 1500 rpm for 5 min to obtain a pellet. The pellet was resuspended in RCME (RNLBIO, SEOUL, KOREA) containing 10% fetal bovine serum (FBS). The cell suspension was re-centrifuged at 1500 rpm for 5 min. The supernatant was discarded and the cell pellet was collected. The cell fraction was cultured overnight at 37 degrees Celcius/5% CO₂ in RCME containing 10% FBS. The adhesion of cells was checked under an inverted microscope the next day. After 24 h, non-adherent cells were removed and the cells were washed with phosphate-buffered saline (PBS). The cell medium was then changed with RKCM (RNLBIO, SEOUL, KOREA) containing 5% FBS. The cells were maintained over four or five days until the cells became confluent, which were represented as passage 0. When cells were 90% confluent, they were subculture-expanded in RKCM until passage 3. The immunophenotype of mesenchymal stem cells was then analyzed using a FACS calibur flow cytometer. Every harvest of mesenchymal stem cells showed a homogenous population of cells with high expression levels of CD73 and CD90 and no expression of CD31, CD34 and CD45. Cell viability evaluated by Trypan blue exclusion method before shipping was >95%. No evidence of bacterial, fungal, or mycoplasmal contamination was observed in cells that tested before shipping. The procedure for mesenchymal stem cells preparation was performed under GMP (Good Manufacturing Practice) conditions (RNLBIO, SEOUL, KOREA).

### Example 2

Restoration of hearing loss using mesenchymal stem cells. A 19 year-old female with total loss of hearing in her left ear and right ear hearing loss (80 ndb) (tube in the tympanic membrane) was intravenously administered (in the arm) autologous mesenchymal stem cells derived from adipose tissue. 200 million cells were administered in one administration, followed by two additional treatments (of 200 million cells each) in one week intervals. The second and third intravenous administrations were also accompanied by 4-10 million cell infusions through the tube in the tympanic membrane. An additional 5 million cells were administered into the right middle ear or inner ear following the last intravenous treatment. The treatment restored 75% of hearing from the right ear.

### Example 3

Treatment of Hashimoto's thyroiditis using mesenchymal stem cells. A 38 year old Asian female patient diagnosed with Hashimoto's thyroiditis and angioedema and urticaria with high antithyroglobulin antibody levels of 343 U/ml was treated. Before treatment, she was on steroid and antihistamine medication. She was intravenously administered (in the arm) 100 million autologous mesenchymal stem cells derived from adipose tissue in one administration. The disease subsided following the single administration. She never had another attack of angioedema and the antibody level declined. Following the first administration, 200 million cells were administered in a second administration. One week later, 200 million cells were administered in a third administration and an additional 200 million cells were administered in a fourth administration one week later. The treatment resulted in decreased antibody levels and cytokines associated with disease. (T3: WNL (within the normal limit); T4: WNL (within the normal limit); TSH: WNL (within the normal limit); FANA: negative; RPR: negative; Anti pyloric IgG: Positive). Consequently, the patient no longer needed to take medication.

### Example 4

Treatment of osteoarthritis using mesenchymal stem cells. A 60 year old white male patient diagnosed with osteoarthritis was intravenously administered (in the arm) autologous mesenchymal stem cells derived from adipose tissue. The patient had a history of renal stones, ulcerative colitis with irritable colon (treated with asacol, prednisolone, pentassa, and balsalazide disodium), hemachromatosis (monitored by ferritin and iron levels), and was undergoing phlebectomy as needed. The histology revealed chronic lymphoplasmacytic colitis and focal acute cryptitis. The patient's history included rheumatic disease (his father and brother were also afflicted), painful joints and use of analgesics, and he had been using a cane for three years to assist with walking; his finger was frozen and unable to draw as an artist as a result of the osteoarthritis and he had 10 bowel movements/day. The treatment consisted of three intravenous injections (200 million cells each). The administrations were separated by one week for each administration. Two to three days following the intravenous administration, 40 million cells were injected into the inter phalangeal joint spaces on both hands (divided amongst the joints). Twelve hours after the local delivery of the stem cells his finger joints were more flexible. Two weeks after treatment, pain diminished markedly. His vision also improved. After one month, the patient was no longer taking any medication and was not using a cane to walk. He could button his shirt in six seconds instead of 10 minutes. He exhibited increased appetite and was able to pursue artistic drawing. He had normal bowel movements (1×/day) and was not taking medication. Shown in Tables 1a and b are the results of a blood test before and after treatment:

TABLE 1a

| | Normal | | Jan/28 | Feb 16 | Feb 28 |
|---|---|---|---|---|---|
| | value | | (before) | (after) | (after) |
| Test items | | | | | |
| Total protein | 6.7-8.3 | (g/dl) | 7.5 | 7.3 | 7.4 |
| albumin | 3.8-5.3 | (g/dl) | 4.2 | 4.2 | 4.3 |
| GOT | 8-38 | (IU/I) | 22 | 29 | 33 |
| GPT | 4-44 | (IU/I) | 12 | 11 | 12 |
| r-GTP | 16-73 | (IU/I) | 42 | 35 | 36 |
| Total | 125-223 | (mg/dl) | 160 | 155 | 152 |
| cholesterol | | | | | |
| Triglyceride | 45-150 | (mg/dl) | 137 | 117 | 113 |
| Renal function | | | | | |
| tests | | | | | |
| BUN | 8-20 | mg/dl | 18 | 19 | 18 |
| Creatinine | | | 1.5 | 1.3 | 1.2 |
| anemia | 0.7-1.3 | mg/dl | | | |
| RBC | 4.3-6.0 | 10⁶/ul | | 3.63 | 3.94 |
| Hemoglobin | 14-18 | g/dl | | 11.8 | 12.8 |
| Inflammatory | | | | | |
| parameters | | | | | |
| WBC | 5.0-10.0 | 10³/ul | | 9.9 | 7.8 |
| CRP(C- | <0.5 | mg/dl | | 1.05 | 0.72 |
| Reactive | | | | | |
| Protein) | | | | | |
| RA | 18 | IU/ml | | 7.6 | 9.8 |
| (Rheumatoid | | | | | |
| Arthritis) factor | | | | | |
| Anti CCP (RA | <5 | U/ml | | 0.8 | 0.7 |
| dx test) | | | | | |

TABLE 1b
(Abnormal Summary)

Comp
Metabolic Panel

| | | | | |
|---|---|---|---|---|
| Alk Phos | 108 | H | U/L | (34-104) |
| Automated Bld | | | | |
| Cnt | | | | |
| RBC | 3.54 | L | 10(12)/L | (4.4-6.0) |
| Hemoglobin | 12.2 | L | g/dL | (14-17) |
| Hematocrit | 36.0 | L | % | (41-51) |
| MCV | 101.7 | H | fL | (800-100) |
| MCH | 34.5 | H | pg | (27-33) |
| Urine | | | | |
| Macroscopic | | | | |
| Specific | <1.005 | L | | (1.005-1.030) |
| Gravity | | | | |

### Example 5

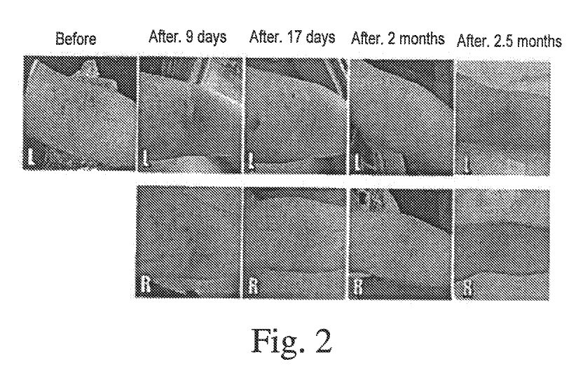

Treatment of atopic dermatitis using mesenchymal stem cells. A 19 year old female patient having long standing, intractable atopic dermatitis with other allergies, including peanut and soybean food allergies, and house dust mite allergy and allergic rhinitis was intravenously administered (in the arm) autologous mesenchymal stem cells derived from adipose tissue. 200 million cells were administered in a first administration, followed by an administration of 200 million cells one week later, following by an additional 200 million cells one week later. In addition to treating the atopic dermatitis (FIG. 2), the allergic rhinitis subsided, as well as other allergies that the patient was experiencing. Her TNF-α and INF-γ serum level decreased as well as her Il-6 and Il-10 level. Her skin became smooth and silky.

### Example 6

Treatment of chronic obstructive pulmonary disease with bronchial asthma using mesenchymal stem cells. A 69 year old Asian male patient diagnosed with chronic obstructive pulmonary disease with bronchial asthma was intravenously administered (in the arm) autologous mesenchymal stem cells derived from adipose tissue. His history included heavy smoking, dust mite, cock roach and aspergillus allergies with coughing, shortness of breath rhinorrhea and increased sputum production. He received 200 million cells in a single administration. One month after treatment, his symptoms improved markedly. His wheezing improved, there was no sputum production, and 40% increment of Fev 1. The 6 minute walk distance test improved of 42 meters. Diffusing capacity remained the same and his breathing and sleep have improved.

### Example 7

Treatment of multiple sclerosis using mesenchymal stem cells. A 55 year old man with multiple sclerosis was treated with mesenchymal stem cells of adipose origin. He received six intravenous injections (about 180 million cells each) and six intrathecal injections (about 20-40 million cells each). The first three injections occurred in weekly intervals, and the final three injections were given seven months later, at weekly intervals. The patient's multiple sclerosis symptoms improved following treatment, and an MRI showed marked improvement.

### Example 8

Autoimmune inner ear disease (AIED) is characterized by progressive, bilateral although asymmetric, and sensorineural hearing loss. Patients with MED have higher frequencies of IFN-γ-producing T cells than the control subject tested. Current therapy for AIED is inadequate. Beyond the low clinical effectiveness, the current therapy for hearing loss (immune based) has limitations because of non-antigen specific nature of these products. Although several antigen specific therapies are in development or in clinical trial in other autoimmune disease, none have yet to be approved as therapies. β-tubulin induces an inflammatory lesion in the inner ear and leads to autoimmune hearing loss, which is orchestrated by CD4 T cells that produce cytokines of the type I profile (Du et al., TUNEL-positive labeling in mouse inner ear caused by tubulin immunization is not apoptosis, ORL, 2003; 17-21; Bin Thou et al. Proceeding of International symposium of Meniere's Disease; Cai et al. ORL J Otorhinolaryngol Relat Spec. 2009; 71(3):135-41. Epub 2009 Apr. 10).

Mesenchymal stem cells (MSCs) (Tai June Yoo. Autologous Adipose Tissue Derived Mesenchymal Stem Cell Intravenous infusions Ameliorate Osteoarthritis (OS), Ulcerative Colitis (UC), Hashimoto Thyroiditis (HT), Atopic Dermatitis (AD) with Allergic Rhinitis, and Chronic Obstructive Pulmonary Disease With Bronchial Asthma. Abst. International Federation of Adipose Tissue Therapeutic Science meeting, Taegi, Korea, October 2009; Zuk et al. Tissue Eng 2001; 7: 211-28; Rasmusson Exp Cell Res 2006; 312:2169-79) are mesoderm-derived cells that reside in the stroma of solid organs and function as precursors of nonhematopoietic connective tissues. Besides their capacity to differentiate into mesenchymal and non-mesenchymal cell lineages and their potential clinical application for the repair of damaged tissues, several recent studies have shown that bone marrow-derived MSCs (BM-MSCs) regulate the immune response, including in vitro inhibition of T cell proliferation, B cell function, and dendritic cell maturation. However, the specific molecular and cellular mechanisms involved in the immunoregulatory activity of BM-MSCs remain a subject of controversy. A critical issue for the clinical translation of BM-MSCs in autoimmunity is that their therapeutic use requires large quantities of cells for infusion, which in most cases, are not available.

Human MSCs can be obtained from subcutaneous adipose tissue (AD-MSCs). Large amounts of human AD-MSCs can be easily obtained from lipoaspirates from healthy donors and rapidly expanded in vitro, and recent studies have reported that human AD-MSCs share some of the immunomodulatory properties that characterize the BM-MSCs.

Importantly, the inventor recently found that human AD-MSCs exert profound suppressive responses on experimental autoimmune hearing loss through human AD-MSCs. Also, stem cell infusion by human cord blood CD133+ cells in mice with noise-induced or drug-induced (by kanamycin) hearing loss repaired hearing loss (Revoltella, Cell Transplant. 2008; 17(6):665-78). The mechanism of immunomodulation is unclear, however it is suggested that Treg cells play a key role in stem cell therapy, but the exact mechanism is still not known (Revoltella, Cell Transplant. 2008; 17(6):665-78; Aggarwal S, Blood 2005 Feb. 15; 105(4):1815-22. Epub 2004 Oct. 19).

The aim of this study is to examine the immunosuppressive activity of human AD-MSCs on β-tubulin-reactive T cells from mice with experimental autoimmune hearing loss (EAHL).

In this study, we will examine whether human AD-MSCs could exert a protective and/or therapeutic role in p-tubulin-induced EAHL in mice and explored the possible mechanism(s) of AD-MSCs in stem cell therapy of autoimmune inner ear disease. Stem cell therapy with AD-MSCs would restore hearing by AD-MSCs's immunomodulating activities, in a non-MHC restricted manner, and IL-10 secretion.

To evaluate the protective effect of human AD-MSCs against the development of autoimmune hearing loss, the mice will be given three i.v. injections of 2×10⁶ human AD-MSCs before the β-tubulin immunization. The therapeutic treatment will begin after the onset of disease after β-tubulin immunization, when EMIL has become well established. Mice with EAHL will be injected i.v. for six times with 2×10⁶ human AD-MSCs. Hearing tests will be performed before and after immunization.

In preliminary studies, systemic infusion of human AD-MSC significantly improved hearing function and restored 100% of hearing in established EAHL mice. Moreover, human AD-MSCs decreased the production of antigen-specific Th1/Th17 cell expansion, and induced the production of anti-inflammatory interleukin-10 in splenocytes. Human AD-MSC also induced the generation of antigen-specific CD4+CD25+FoxP3+ Treg cells.

At six weeks of age BALB/c mice were immunized subcutaneously with 300 μg of β-tubulin emulsified with an equal volume of CFA containing 2 mg/ml of H37Ra Mycobacterium tuberculosis. The mice were given boosters by subcutaneous injection with 300 μg of β-tubulin emulsified with ICA twice at 1-week intervals, 2 weeks after initial immunization.

Two weeks later, all β-tubulin-immunized-mice succumb to a significant increase in ABR click and pure tone thresholds at all frequencies tested from 8 kHz to 32 kHz. After that, mice with hearing loss were injected i.v. for 6 times with 2×10⁶ human AD-MSCs or PBS to determine the efficacy of human AD-MSCs on disease progression in mice with already established EAHL.

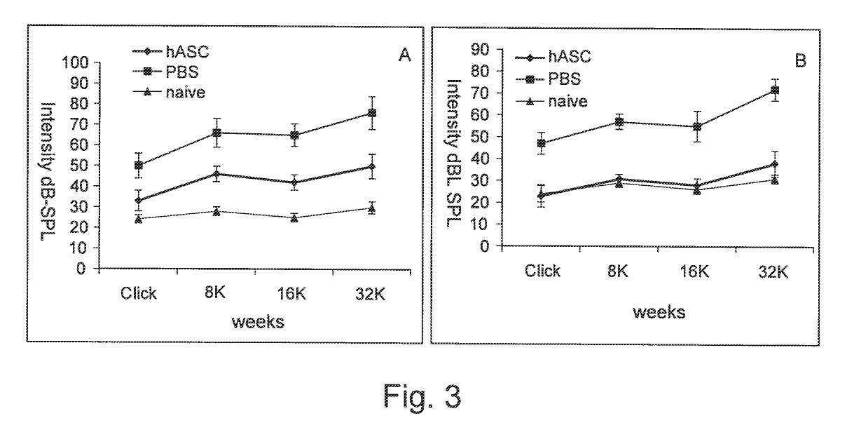

After three injections, mice with EAHL significantly decreased ABRs (FIG. 3A) at the all frequencies tested in comparison with PBS controls; however, after six injections of hASCs, β-tubulin immunized mice restored their hearing (FIG. 3B), similar to naïve normal hearing mice.

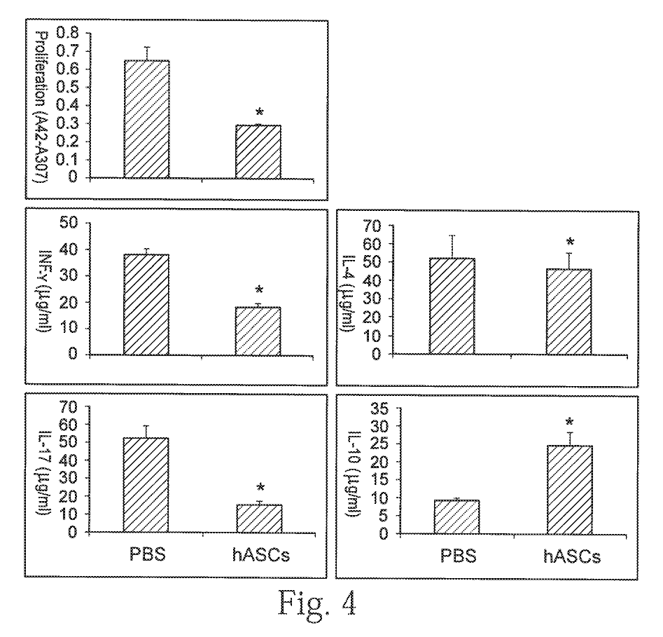

Splenocytes from the mice that were administered human AD-MSCs during the ongoing immune process produced significantly lower levels of IL-17 and IFN-γ than did cells from mice administered PBS (FIG. 4). Moreover, human AD-MSCs dramatically stimulated the production of IL-10 (FIG. 4) by β-tubulin-activated T cells, whereas the Th2-type cytokine IL-4 was not significantly affected.

Thus, these findings indicate that administration with human AD-MSCs in therapeutic regimens to mice with EAHL was associated with strong immune-modulating effects on the priming of β-tubulin-specific CD4⁺ T cells, resulting in skewing of activated CD4 T cells toward lower activity of Th1 and Th17 effector cells-inhibit the differentiation of auto-reactive Th1 cells, but increased activity of the anti-inflammatory cytokine IL-10, suggesting that this treatment can generate IL-10-secreting Treg cells.

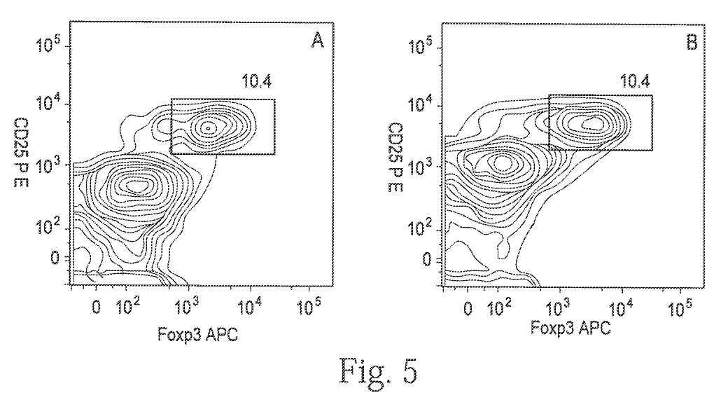

In addition, administration of human AD-MSCs had significantly higher numbers of CD4⁺CD25⁺FoxP3⁺ Treg cells in splenocytes (FIG. 5A) than did PBS control mice (FIG. 5B), indicating human AD-MSCs could be inducing Treg cells secreting IL-10, which suppresses the self-reactive T cells.

### Example 9

### Administering Human Adipose-Derived Mesenchymal Stem Cells to Prevent and Treat Experimental Arthritis

Rheumatoid arthritis is a chronic, systemic, inflammatory disease primarily targeting the synovium and affecting approximately 1% of the population. Human adipose-derived mesenchymal stem cells (hASCs) were recently found to suppress effector T cell and inflammatory responses and, thus, to have beneficial effects in various autoimmune disorders. In this study, we examined whether hASCs could exert a protective and/or therapeutic role in collagen-induced arthritis (CIA) in mice and explored the possible mechanism(s) of hASCs in stem cell therapy of rheumatoid arthritis.

### Methods

Clinical efficacy was tested in DBA/1 mice with CIA that were administered hASCs before or during arthritis induction. Inflammatory response was determined by measuring the levels of different inflammatory mediators in the joints and serum. The Th1-mediated autoreactive response was evaluated by determining the proliferative response and cytokine profile of splenocytes stimulated by the autoantigen. The frequency of regulatory T (Treg) cells and their suppressive capacity on self-reactive Th1 cells were also determined.

### Results

hASCs both prevented and treated CIA by significantly reducing the incidence and severity of experimental arthritis. We further demonstrated that treatment with hASCs inhibited the production of various inflammatory cytokines and chemokines, decreased antigen-specific Th1/Th17 cell expansion, and induced the production of anti-inflammatory interleukin-10 in splenocytes and joints. Moreover, hASCs could induce the generation of antigen-specific Treg cells with the capacity to suppress collagen-specific T cell responses.

The present work demonstrated hASCs as key regulators of immune tolerance with the capacity to suppress autoimmune and inflammatory responses and induce the generation of Treg cells.

### Example 10

### Stem Cell Therapy for Hearing Loss:Suppression of Auto-Reactive T Cell Responses

Autoimmune inner ear disease (AIED) is characterized by progressive, bilateral although asymmetric, and sensorineural hearing loss. Patients with AIED have higher frequencies of IFN-γ-producing T cells than the control subject tested. Adult mesenchymal stem cells were recently found to suppress effector T cell and inflammatory responses, and thus to have beneficial effects in various immune disorders. The aim of this study is to examine the immunosuppressive activity of human adipose-derived MSCs (hASCs) on β-tubulin-reactive T cells from mice with experimental autoimmune hearing loss (EAHL).

### Methods

Female BALB/c mice underwent β-tubulin immunization to develop EAHL, mice with EAHL were administered hASCs or PBS intraperitoneally, once a week for six consecutive weeks. Auditory brainstem responses (ABR) were examined over time. The Th1-mediated auto-reactive response was evaluated by determining the proliferative response and cytokine profile of splenocytes stimulated with the autoantigen.

### Results

Systemic infusion of hASCs significantly improved hearing function and protected hair cells in established EAHL. Moreover, hASCs decreased the production of antigen-specific Th1/Th17 cell expansion, and induced the production of anti-inflammatory interleukin-10 in splenocytes.

The present work demonstrated that hASCs as key regulators of immune tolerance, with the capacity to suppress auto-reactive T cells.

### Example 11

### The Effect of Adipose Stem Cell on Survival in a Model of Graft Versus Host Disease

Graft-versus-host disease (GvHD) and graft rejection have remained major problem for transplantation. Human adipose-derived mesenchymal stem cells (hASCs) were recently found to suppress effector T cell and inflammatory responses, and thus to have beneficial effects in GvHD. The goal of the study was to investigate the immunoregulatory properties of these cells, and evaluated their capacity to control GVHD in mice.

### Methods

Following irradiation, the mice were injected IV via tail vein with 20×10⁶ single donor PBMC in approximately 50 μl of base media. Following PBMC injections, the mice received weekly IV injections of hASCs at 500,000 cells per 100 μl tail vein injection. Survival was assessed by the righting reflex; at sacrifice a gross necropsy was conducted, and spleens were harvested and weighed prior to snap freezing. Terminal blood samples were collected and processed to serum.

### Results

hASCs significantly increased the survival of experimental GvHD mice. Treatment with hASCs decreased Th-1/Th-17 cell expansion, and induced the production of anti-inflammatory interleukin-10 in splenocytes. Moreover, hASCs could keep the body weight of GvHD mice.

The present work demonstrated hASCs as key regulators of immune tolerance, with the capacity to suppress Th-1/Th-17 responses, and increase the survival rate of GvHD mice.

### Example 12

### The Effect of Mesenchymal Stem Cells in an Animal Model of Arthritis

Rheumatoid arthritis (RA) is an autoimmune disease caused by loss of immunologic self tolerance that leads to chronic inflammation in the joints and subsequent cartilage destruction and bone erosion. The crucial process underlying disease initiation is the induction of autoimmunity to collagen-rich joint components; later events evolve a destructive inflammatory process (Firestein Nature 2003; 423: 356-61). Type 2 collagen induced arthritis (CIA) was developed in Memphis by Kang et al (J Exp Med. 1977 Sep. 1; 146(3):857-68) and it is widely used as inflammatory arthritis model for RA. Progression of the autoimmune response implies the development of autoreactive Th1 and Th17 cells, their entry into the joint tissues, and their release of proinflammatory cytokines and chemokines, which promote macrophage and neutrophil infiltration and activation. Excessive levels of mediators of inflammation, such as cytokines, free radicals, and extracellular matrix-degrading enzymes, produced by infiltrating inflammatory cells play a critical role in joint damage (Firestein Nature 2003; 423: 356-61; Yoo T J et al. J Exp Med. 1988 Aug. 1; 168(2):777-82).

Mesenchymal stem cells (MSCs) are mesoderm-derived cells that reside in the stroma of solid organs and function as precursors of nonhematopoietic connective tissues. Besides their capacity to differentiate into mesenchymal and nonmesenchymal cell lineages (Pittenger et al. Science 1999; 284: 143-7; Zuk et al. Tissue Eng 2001; 7: 211-28) and their potential clinical application for the repair of damaged tissues, several recent studies have shown that bone marrow-derived MSCs (BM-MSCs) regulate the immune response, including in vitro inhibition of T cell proliferation, B cell function, and dendritic cell maturation (Rasmusson 1. Exp Cell Res 2006; 312: 2169-79; Nauta et al. Blood 2007; 110: 3499-506; Bartholomew et al. Exp Hematol 2002; 30: 42-8; Glennie et al. Blood 2005; 105: 2821-7; Beyth et al. Blood 2005; 105: 2214-9). The immunomodulatory effects of stem cells are probably due to Treg cells and other cytokines.

Human MSCs obtained from subcutaneous adipose tissue (AD-MSCs) have recently emerged as a potentially attractive alternative source of MSCs (Nauta et al., Blood 2007; 110: 3499-506; Bartholomew et al. Exp Hematol 2002; 30: 42-8; Beyth et al. Blood 2005; 105: 2214-9; Yanez et al., Stem Cells 2006; 24: 2582-91). Large amounts of human AD-MSCs can be easily obtained from lipoaspirates from healthy donors and rapidly expanded in vitro to generate a clinically effective dosage, and recent studies have reported that human AD-MSCs share some of the immunomodulatory properties that characterize the BM-MSCs (Zheng et al., Rheumatology (Oxford) 2008; 47: 22-30; Puissant et al., Br J Haematol 2005; 129: 118-29).

Adult human mesenchymal stem cells (MSCs) were recently found to suppress effector T cell responses and to have potential beneficial effects in various immune disorders. The purpose of this study is to examine a new protective and therapeutic strategy for collagen-induced arthritis, an animal model for RA, based on the administration of human adipose-derived MSCs (AD-MSCs).

A study in an animal model of arthritis, namely collagen induced arthritis (CIA) in DBA/1LacJ mice, is proposed to assess if stem cell therapy will provide an effective therapeutic for CIA in mice. To evaluate the protective effect of human AD-MSCs against the development of CIA, the mice will be given three i.v. injections of 2×10⁶ human AD-MSCs before the CII immunization. The therapeutic treatment will begin after the onset of disease, when arthritis had become well established. Mice with CIA will be injected i.v. for six days with 2×10⁶ human AD-MSCs, PBS, or 2×10⁶ Jurkat cells. PBS and Jurkat cells treated mice will serve as control groups. Arthritis severity will be assessed by clinical scoring and measurement of hind paw thickness.

In preliminary studies, hASCs both prevented and treated CIA by significantly reducing the incidence and severity of experimental arthritis. Treatment with hASCs inhibited the production of various inflammatory cytokines and chemokines, decreased antigen-specific Th1/Th17 cell expansion, and induced the production of anti-inflammatory interleukin-10 in splenocytes and joints. Moreover, hASCs could induce the generation of antigen-specific Treg cells with the capacity to suppress collagen-specific T cell responses.

### Results

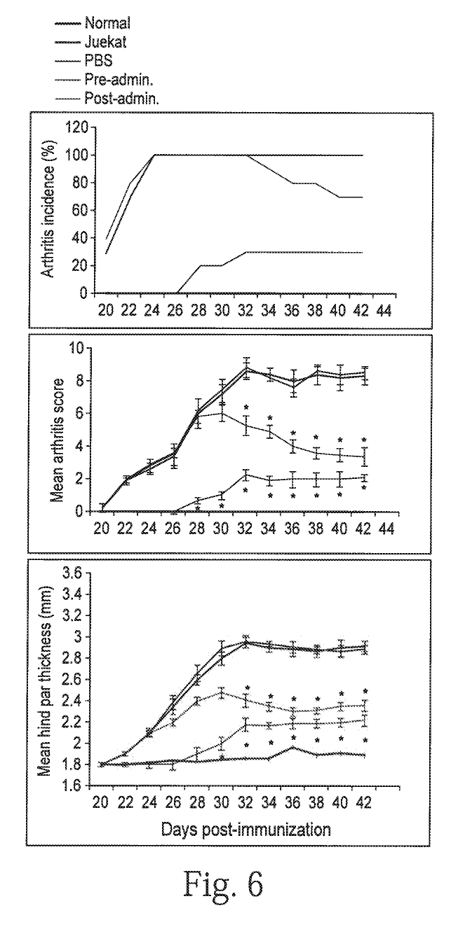

DBA/1LacJ mice were immunized with 100 μg of chicken type II collagen and 100 μg of Mycobacterium tuberculosis H37Ra subcutaneously into the base of the tail on day 0. To evaluate the protective effect of human AD-MSCs against the development of CIA, the mice were given three i.v. injections (days −9, −7 and −4) of 100 μl of PBS containing 2×10⁶ human AD-MSCs before the CII immunization (see Violet color in FIG. 1a, 1b, 1c). The therapeutic treatment was begun after the onset of disease, when arthritis had become well established (arthritis score >2). Mice with CIA were injected i.v. for three days (days 26, 28 and 32) with 2×10⁶ human AD-MSCs, PBS, or 2×10⁶ Jurkat cells. PBS and Jurkat cells treated mice served as control groups. Arthritis severity was assessed by clinical scoring and measurement of hind paw thickness (see light blue color (post-admin) in FIG. 6).

CII-immunized mice first displayed visible arthritic signs characterized by edema and/or erythema in paws around day 20 after immunization, and showed maximum paw swelling by day 32, which gradually diminished thereafter. However, treatment with human AD-MSCs before the onset of arthritis (days −9, −7 and −4) resulted in a marked decrease in the incidence of arthritis, with 70% of the treated mice free of clinical arthritis at the end of the observation period, and mice with CIA significantly reduced paw swelling throughout disease progression of arthritis.

To determine the efficacy of human AD-MSCs on disease progression in mice with already established arthritis, treatment of immunized mice was delayed until mice developed overt arthritis (arthritis score >2), and the mice then received either PBS, Jurkat cells or human AD-MSCs three times consecutively on alternate days. Human AD-MSCs progressively attenuated the severity of the clinical signs and hind paw volume of the arthritic mice, and significantly decreased the percentages of mice with arthritis, with 40% of the treated mice free of clinical arthritis at the end of the observation period, as compared to the PBS and Jurkat cells-treated arthritic mice. We next investigated the mechanisms underlying the decrease in severity of CIA by protective and therapeutic administration of human AD-MSCs. In the protective approach, assays performed 42 days after the last injection of human AD-MSCs, still revealed significantly decreased levels of various inflammatory cytokines and chemokines. Human AD-MSC injection significantly reduced protein expression of various inflammatory cytokines (IL-1α, IL-1β, IL-6, IL-12, IL-17, TNF-α, and IFN-γ) and chemokines (MCP-1, Rantes, and KC), while it increased expression of the antiinflammatory cytokine IL-10, in the joints of mice with CIA.

In the therapeutic approach assays were performed 10 days after the last treatment with the human AD-MSCs. Human AD-MSC injection significantly reduced protein expression of various inflammatory cytokines (IL-1α, IL-1β, IL-6, IL-12, IL-17, TNF-α, and IFN-γ,) and chemokines (MCP-1, Rantes, and KC). We also found that human AD-MSCs in the therapeutic treatment protocol significantly increased the antiinflammatory cytokine IL-10, in the joints of mice with CIA.

Levels of the inflammatory cytokines and chemokines in the blood serum were determined on day 42 for the prophylactic treatment on days −9, −7 and −4 and the therapeutic treatment on days 26, 28 and 32 after the immunization with CIA, respectively. Consistent with the joint swelling, the levels of IL-1α, IL-6, IL-17, MCP-1, Rantes, and KC in the PBS-treated CIA mice were systemically overproduced in the serum.

In contrast, markedly low serum levels of IL-1α, IL-6, IL-17, MCP-1, Rantes, and KC were seen in the CIA mice treated either treated prophylactically with human AD-MSCs or therapeutically with human AD-MSCs. Therefore, the broad antiinflammatory activity of human AD-MSCs in the inflamed joint was accompanied by down-regulation of the systemic inflammatory response (see Tables 2 and 3).

TABLE 2
The production of mediators of inflammation in the joint extracts

| | TNFα | IL-6 | IL-12 | IFNγ | MCP-1 | Rantes |
|---|---|---|---|---|---|---|
| PBS | 190.6 ± 34 | 1551 ± 283.3 | [symbol] 77.8 ± 19.6 | 388.8 ± 18 [symbol] | 22633.3 ± 1396.1 | [symbol] 020. [symbol] ± 125.2 |
| Pre-admin. | 31.6 ± 5.1 | 249 ± 31.6 | 42.5 ± 3.7 | 140.4 ± 21 6 | [symbol] 5246.6 ± 2950 | 2165 ± 6 [symbol] .9 |
| Post-admin. | 19.5 − 5.5 | 150.2 + 80.6 | 29.6 + 8.7 | 54 + 1 [symbol] 8 | 8509.2 + 3363 | 1163.3 + 278.8 |

| | IL-1α | IL-1β | IL-10 | IL-17 | KC |
|---|---|---|---|---|---|
| PBS | 168.3 ± 13.7 | 20 ± 2.3 | 20.76 ± 5.4 | 6434.3 ± 728.6 | 789.6 ± 157.9 |
| Pre-admin. | 44.8 ± 10.9 | 10.6 ± 2.6 | 32.7 ± 0.6 | 520.3 ± 103.7 | 305 ± 56.3 |
| Post-admin. | 30.4 ± 11.4 | 6.4 ± 1.5 | 368.6 ± 129.8 | 103.7 ± 49.2 | 232.2 ± 26.6 |

[symbol]

indicates data missing or illegible when filed

TABLE 3
The production of mediators of inflammation in the serum

| | IL-6 | MCP-1 | Rantes | IL-lα | IL-17 | KC |
|---|---|---|---|---|---|---|
| Control | 361.5 ± 100.8 | 22.3 ± 1.5 | 306 ± 93.2 | 399.7 ± 40.3 | 53 ± 17.6 | 1203.8 ± 483.9 |
| hASC-Pre | 105.7 ± 55.8 | 11.3 ± 1.2 | 220.3 ± 48.7 | 170.7 ± 32.9 | 19.7 ± 3.1 | 1057.6 ± 475.3 |
| hASC-The | 70.8 ± 7.3 | 10 ± 0 | 150.1 ± 44.7 | 138.5 ± 46.3 | 14.7 ± 4.1 | 388 ± 124 |

1. A method of treating or preventing a disease in a patient comprising intravenously administering a therapeutically effective amount of mesenchymal stem cells to the patient, wherein the disease is selected from the group consisting of osteoarthritis, rheumatoid arthritis, multiple sclerosis, stroke, ulcerative colitis, Hashimoto's thyroiditis, psoriasis, atopic dermatitis, allergic rhinitis, and chronic obstructive pulmonary disease with bronchial asthma.

2. The method of claim 1, wherein the mesenchymal stem cells are autologous.

3. The method of claim 2, wherein the mesenchymal stem cells are isolated from the patient's adipose tissue.

4. The method of claim 3, wherein the disease to be treated is osteoarthritis.

5. The method of claim 3, wherein the administration comprises three intravenous injections of 200-300 million cells each.

6. The method of claim 3, wherein the administration comprises three intravenous injections of 200 million cells each and an injection of 40 million cells divided amongst the inter phalangeal joint spaces in the patent.

7. The method of claim 1, wherein the disease is ulcerative colitis.

8. The method of claim 1, wherein the disease is Hashimoto's thyroiditis.

9. The method of claim 1, wherein the disease is allergic rhinitis.

10. The method of claim 1, wherein the disease is chronic obstructive pulmonary disease with bronchial asthma.

11. A method of treating hearing loss in a patient, comprising intravenously administering a therapeutically effective amount of mesenchymal stem cells to the patient.

12. A method of treating hearing loss in a patient, comprising administering a therapeutically effective amount of mesenchymal stem cells to the patient.

13. The method of claim 12, wherein the cells are isolated from adipose tissue.

14. The method of claim 12, wherein the hearing loss is autoimmune hearing loss.

15. The method of claim 12, wherein the hearing loss is drug-induced hearing loss.

16. The method of claim 12, wherein the hearing loss is due to injury.

17. The method of claim 12, wherein the administration comprises injection of cells into the patient's ear.

18. The method of claim 11, wherein the administration comprises three intravenous injections of 200 million cells each.

19. The method of claim 18, wherein the administration further comprises local administration of mesenchmal stem cells to the inner or middle ear.
